(19) United States
(12) Patent Application Publication
(10) Pub. No.: US 2008/0146881 A1
(43) Pub. Date: Jun. 19, 2008

(54) Retractor-Retainer of Intestines for Celioscopic Surgery

(75) Inventors: Yves Alimi, Marseille (FR); Frederic Mouret, Marseille (FR); Joachim Ramos Clamote, Marseille (FR)

Correspondence Address: YOUNG & THOMPSON, 209 Madison Street, Suite 500, ALEXANDRIA, VA, 22314, US

(73) Assignees: PROTOMED, Marseille Cedex (FR); UNIVERSITE DE LA MEDITERRANEE, Marseille Cedex 07 (FR); ASSISTANCE PUBLIQUE HOPITAUX DE MARSEILLE, Marseille (FR)

(21) Appl. No.: 11/722,734

(22) PCT Filed: Dec. 21, 2005
(86) PCT No.: PCT/EP05/57028

§ 371 Date: Jun. 25, 2007

(30) Foreign Application Priority Data
Dec. 24, 2004 (FR) 04/13885

(51) Int. Cl. A61B 17/02

(52) U.S. Cl. 600/204

(57) ABSTRACT

A retractor-retainer of intestines for celioscopic surgery, includes: a guide wire having a proximal portion consisting of a handle adapted to be connected to an operating table, an elongated distal portion designed to be introduced into the abdominal cavity during a celioscopic procedure; an elongated support mandrel along which is fixed a contention net, folded in such a way as to enable it to be deployed in situ; the distal end of the support mandrel and the distal portion of the guide wire being provided with complementary fast coupling elements; and an instrument for inserting the support mandrel and the contention net into the abdominal cavity and fixing it on the guide wire, the instrument including a barrel whereof at least the distal portion is hollow and dimensioned to receive the support mandrel and the contention net connected thereto.

The present invention relates to an intestinal retractor or retainer used in celioscopic surgery. This is an invasive surgical instrument for temporary use in video-assisted surgery. More precisely, the device has the particular function of retaining the intestinal loops outside the field of operational vision during surgical operations in the abdomen, and especially abdominal aortic restoration surgery, and of enabling the intervention to take place without risk of contact with the isolated internal organs. This device can for instance be used in digestive and vascular surgery to dissect and repair organs or vessels. It limits the time during which the patient is in an uncomfortable position that could itself be the source of complications, in particular respiratory.

Each year, more than one hundred thousand patients worldwide benefit from abdominal aortic restoration. Conventional surgical procedures involve a degree of morbidity and mortality due, in part at least, to the large abdominal opening, from 25 to 35 cm long. Video-assisted mini-invasive procedures have been used for more than 20 years in gynecological and digestive surgery, but are of more recent use in aortic surgery. This is due to the particular difficulties involved in the dissection of a deep organ and the risks of major bleeding in the event of error.

One of the first obstacles to reaching the aorta is the presence of the intestinal loops which have to be moved out of the operative field during surgery. There are few laparoscopic procedures available from other developers of vascular surgery. Nevertheless, two main trends are apparent. The first consists in placing the patient on the side in a strongly inclined position in order to rock the intestines so that the aorta appears. Unfortunately, the aorta is no longer accessible directly and the operating procedure is more difficult to acquire. The results obtained with this procedure are encouraging, but, in spite of considerable experience, they are never quite as good as those achieved with the conventional surgery (in terms of morbidity, mortality, conversion, etc). Nevertheless, these results may be considered disappointing as the characteristic of a new procedure is not to simply emulate but to make a real improvement for the patient.

Another approach is to seek direct access to the aorta without placing the patient in an uncomfortable tilted position for several hours. This requires use of an intestinal retractor. Several systems have been developed. One of them consists of a retractor comprising a square net folded in two and sewn so as to form a “small pocket”. Two openings are arranged inside this pocket, at two ends of the same side. These openings allow the passage of rakes conventionally used in open surgery. These two rakes are used to retract the intestines on each side of the pocket.

This retractor presents several disadvantages. It requires two incisions, one for each rake. It is not easy to install because the net and the two rakes have to be introduced through three different openings then, once in place, each rake is inserted inside the net. It has to be held in position by hand since it is not sufficiently rigid for “definitive” installation throughout the operation. Lastly, it does not allow the intestines to be retracted correctly in its lower part and the latter can pass “underneath” and congest the operative field. Indeed, in the absence of a rigid structure in its lower part, this can become deformed and allow passage of the intestines.

Document FR 2805731 A1 also describes a retractor equipped with a net for positioning and retracting the internal intra-peritoneal organs. This net can be fixed onto the abdominal wall by two stitches so as to form a hammock-like arrangement. Such a configuration does not offer any accuracy of placement or element of rigidity.

Another system proposed for the laparoscopic surgery in the vascular field consists of a retractor with five arms already used in digestive surgery, but which presents serious disadvantages. It does not allow all the intestinal loops to be retracted, and therefore has to be retracted permanently by hand in order to free the operative field. In this case, the surgeon, or one of his assistants, is, prevented from using one hand.

To avoid the hemorrhage risks associated with these “closed” laparoscopic procedures, a procedure known as “Hand-Port” has been proposed. This allows the surgeon to insert the non-dominant hand into the patient's abdomen, through a 7 to 8 cm long incision, while creating a pneumoperitoneum at a constant pressure of 14 mmHg using a system of tight sleeve. This hand is used to retract the internal organs and compress a vessel in the event of a sudden hemorrhage; the surgeon carries out the aortic dissection and the aortic anastomoses with the other hand, under the control of a video camera held by an assistant. This technique was abandoned for several reasons. First of all, the surgeon has one hand occupied retracting the internal organs and, even if this system is reassuring for the surgeons beginning to use this technique, it does not represent a move towards completely laparoscopic surgery.

The intestinal retractor used in vascular surgery is intended for use on fragile and old patients (often, and increasingly, from 80 to 90 years old), much more so than in digestive surgery (50 to 60 years old). Therefore, the techniques used in the two latter surgical procedures for retracting the intestines involve more risk for the patients. Indeed, gravity is generally used to retract the intestines by placing the patient in a right lateral decubitus and Trendelenbourg position (head downwards). Moreover, the aorta is very deeply positioned in the abdomen and more median. This requires extreme inclination and this could lead to the complications already referred to, as well as cerebral oedemas (work by Barbosa), hence the usefulness of the intestinal retractor.

Some retractors have been proposed to free the field of vision inside the abdominal cavity during a laparoscopic procedure.

Document U.S. Pat. No. 5,465,711 describes an inflatable intestinal retraction device that can be used in endoscopic surgery. This inflatable retractor allows the intestines to be retracted in order to access organs such as the spinal column, aorta, kidneys, etc. In certain applications, such as abdominal aortic surgery, this retractor has the disadvantage of not allowing the surgeon to adapt it in situ to the patient's anatomy. It has a predefined form and is inflated so as to adopt a predefined shape and volume allowing access to the ill organ.

Document US-2003/0004473 described a retainer of deformable internal organs consisting of a rectangular inflatable envelope which is essentially flat, in which are placed sheets, some of which at least are in a malleable material. Such a cumbersome device can only be used in open surgery and not, of course, in invasive microsurgery.

Document U.S. Pat. No. 5,318,586 describes a device that can be used in laparoscopic surgery. This device can be used to retract the intestines or other organs. It includes a retractor consisting of an inflatable end-piece housed, with a sliding possibility, in a rigid tube intended to be introduced into the abdomen. This end-piece can be pushed out of the distal end of the rigid tube and dilated by means of a gas or a fluid, the aforementioned end-piece having various forms and sizes depending on the interventions which the device is used for. The inflatable end-piece has, in all the cases, a predetermined form and the insertion and guidance tube is rigid, so that the device cannot be adapted in situ to the anatomy of the patient.

Examination of the methods and devices disclosed by the state of the art allows us to observe that the need for a device which can be easily adapted “in situ” to any surgical approach, prevents displacement of the intestines once positioned, and releases the surgeon from any constraints during an operation, has not yet been developed.

The invention proposes a retractor and retainer of intestines for use in laparoscopic surgery in order to overcome the problem of the inadequacy to the patient's anatomy encountered with the state of the art devices, and which can be installed in a short period of time.

This objective is achieved by the invention thanks to a device comprising:

a guiding mandrel with a proximal portion consisting of a handle that can be connected to an operating table, a distal portion with elongated form which is introduced into the abdominal cavity of the patient during laparoscopic surgery;

a support mandrel of elongated form on which and along which is fixed a contention membrane such as a net folded up according to a folding method allowing it to deploy in situ, the distal end of this support mandrel and the distal portion of the guiding mandrel being equipped with quick complementary means for fast coupling;

and an instrument for the insertion of the support mandrel and the contention net into the abdominal cavity, and fixing of the aforesaid support mandrel on the guiding mandrel, this instrument comprising a barrel, the distal portion at least of which is hollow and is dimensioned to receive the aforementioned support mandrel and the contention net connected to the latter.

According to a preferred embodiment, the distal portion of the guiding mandrel consists of a tubular guide housing a coupling rod, such rod having the capacity to move axially in the said tubular guide whose distal end is equipped with a transversal passage, the distal end of the support mandrel comprising a lug intended to form part of the aforementioned transverse passage and provided with a lug or transversal notch into which the distal end of the coupling rod can be engaged so as to fix the support mandrel to the guiding mandrel.

According to a preferred embodiment, the coupling rod is subjected to the action of a spring tending to push it back into a coupling position, so that the support mandrel is fixed to the guiding mandrel automatically by simply sinking the coupling lug of the said support mandrel into the transversal passage of the said guiding mandrel.

According to another embodiment, the handle of the guiding mandrel is equipped with a push-button control mechanism so that by successively pressing on this push-button, alternately, either the distal end of the coupling rod is inserted into the transversal notch of the coupling lug of the support mandrel or the said rod is withdrawn.

The device according to the invention offers interesting advantages; during abdominal aortic restoration surgery it allows in particular:

perfect in situ adaptation to the anatomy of the patient;

stable fixing to the operating table: it is maintained in position once for all, so that the surgeon's and the assistants' hands are free and do not have to be occupied keeping the operative field free;

installation in a relatively short period of time for this type of surgery, i.e. around 15 to 20 minutes;

direct access to the abdominal aorta, thus avoiding the large retroperitoneal dissections;

creation of a barrier to passage of the intestinal loops throughout the intervention;

the patient to be maintained in dorsal decubitus (flat on the back), avoiding prolonged Trendelenbourg positions at 25° (head down) and in right dorsal decubitus position between 25 and 60° (patient lying on the right side), which can have harmful effects for old patients with a defective cardiorespiratory function (atelectasis of the pulmonary apexes, ophthalmic and cerebral oedemas were noted with certain methods currently applied);

a reduction of the pneumoperitoneum pressure (gas blown into the stomach) from 14 to 8 mm Hg, with a reduction of digestive and renal repercussion (capillary ischemia on the splanchnic and oligo-anuria territory).

The above aims, characteristics and advantages, and others, will better be revealed by the description which follows and the annexed drawings in which:

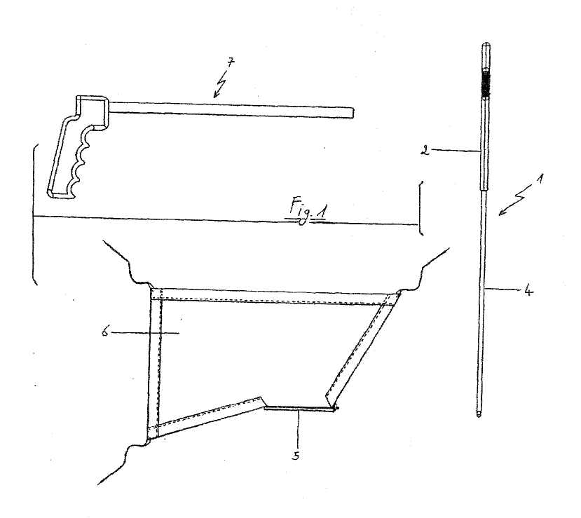

FIG. 1 is a view illustrating, separately, the component parts of the intestinal retractor-retainer device according to the invention.

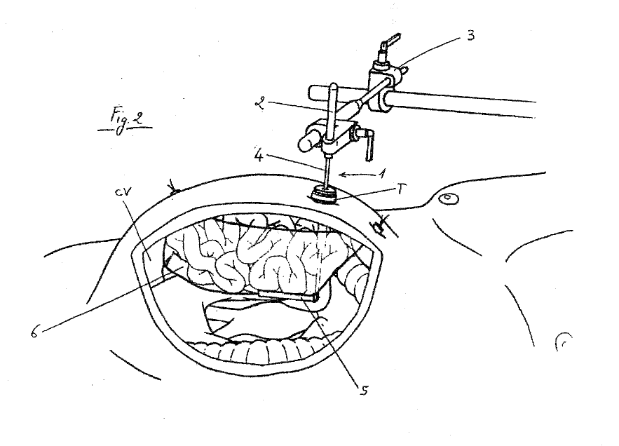

FIG. 2 is a view in perspective showing the retractor-retainer according to the invention, the invasive parts of which are placed in the abdominal cavity of a patient and the external part of which is connected to a device fastening it to an operating table partially shown.

FIG. 3 is an axial sectional view of a first example of the embodiment of the guiding mandrel.

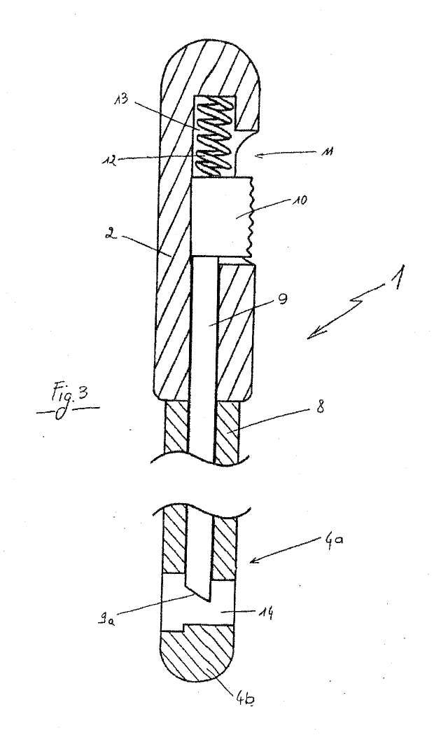

FIG. 4A is a view in perspective and from the top front of the support mandrel.

The FIG. 4B is a view in perspective and from the top rear of this support mandrel.

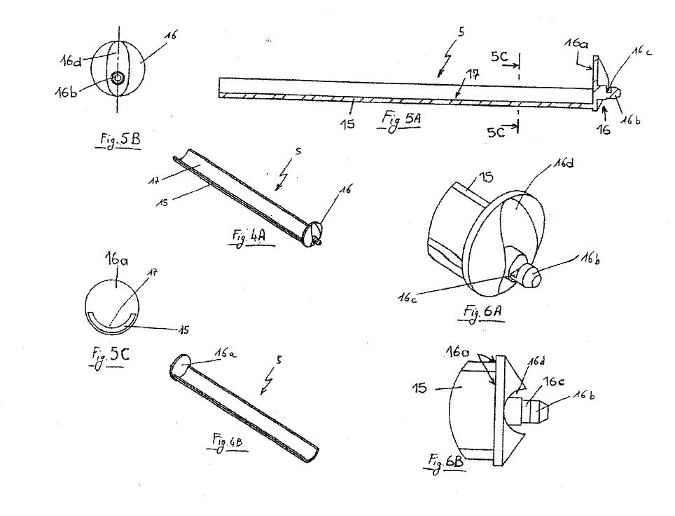

FIG. 5A is a longitudinal sectional view of the said mandrel.

FIG. 5B is a front view of the coupling head of the latter.

FIG. 5C is a sectional view along line 5C-5C on FIG. 5A.

FIG. 6A is a perspective view of the coupling head of the support mandrel.

FIG. 6B is a top view of FIG. 6A.

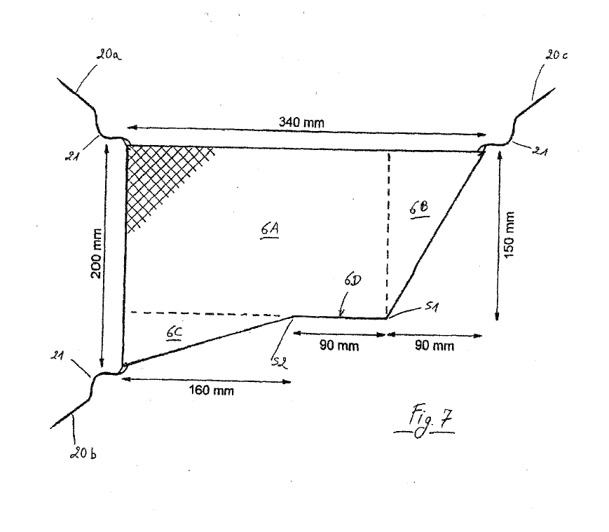

FIG. 7 is a front view of a contention net in a deployed state.

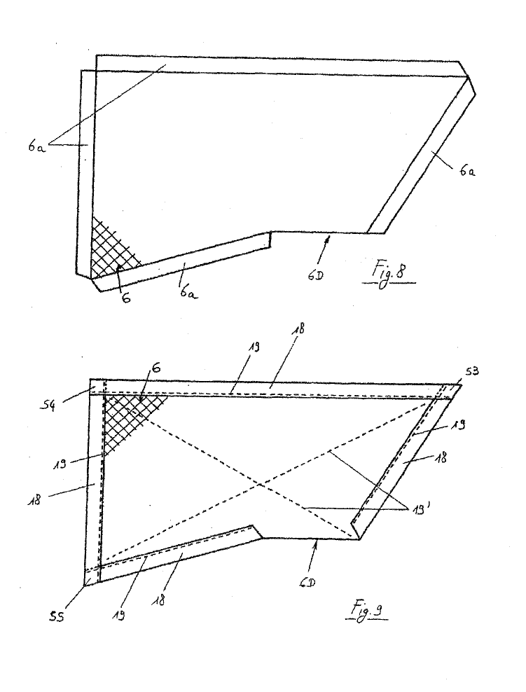

FIGS. 8 and 9 are views illustrating two stages in the execution of a reinforced edge of the net.

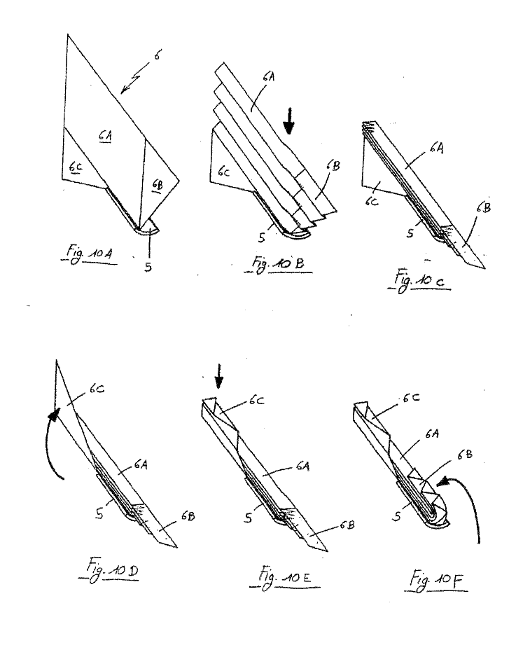

FIGS. 10A to 10F illustrate the contention net folding states allowing its introduction into the insertion instrument barrel,

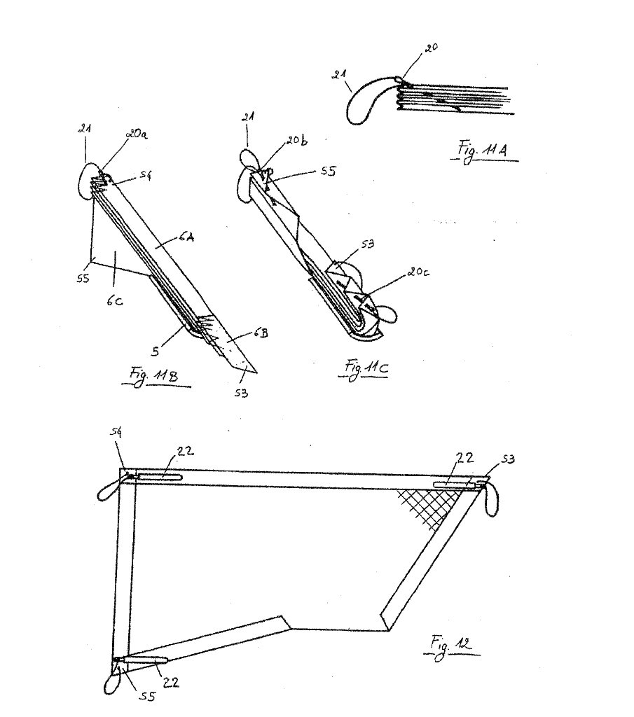

FIG. 11A is a partial view and FIGS. 11B and 11C are views in perspective illustrating the positioning of the fixing needles in the folded contention net.

FIG. 12 illustrates another method of positioning the fixing needles on the net shown in a deployed state.

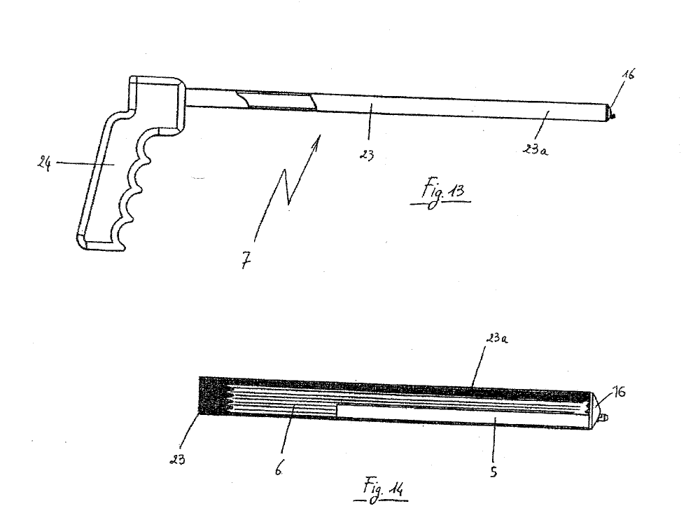

FIG. 13 is an elevation view, with part sectional view, of the support mandrel and the contention net insertion instrument.

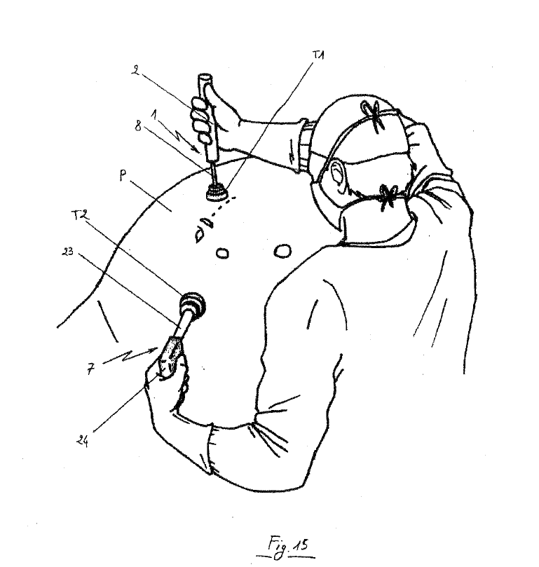

FIG. 14 is a longitudinal sectional and larger scale view of the distal portion of the insertion tube in which are positioned the support mandrel and the contention net.

FIG. 15 is a view in perspective showing the insertion of the guiding mandrel and the mandrel supporting the net, in the abdominal cavity.

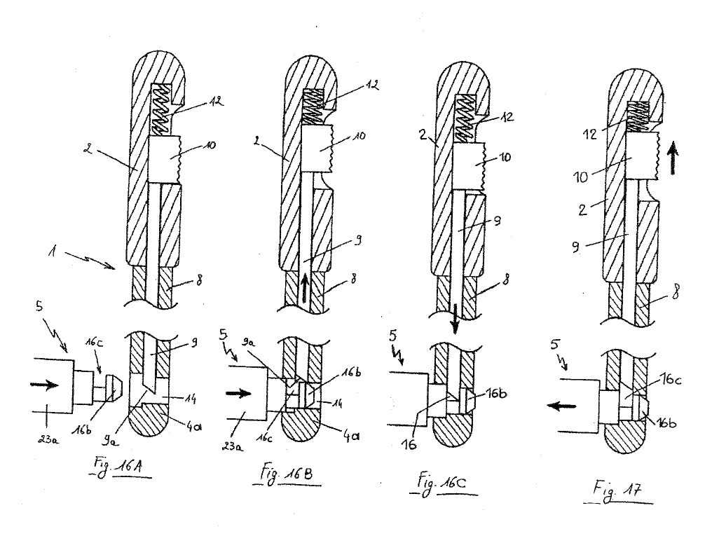

FIGS. 16A, 16B, 16C illustrate, by axial sectional views, the method of clipping the support mandrel on the guiding mandrel.

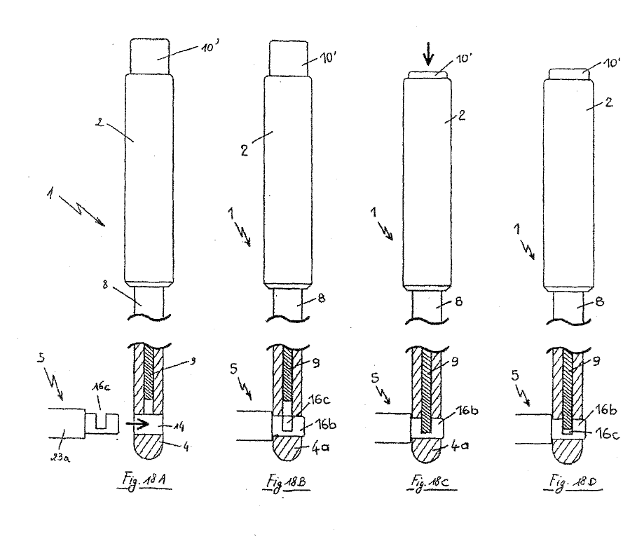

FIG. 17 is an axial sectional view illustrating the separation of the support mandrel and the guiding mandrel at the end of the intervention.

FIGS. 18A to 18D illustrate a method of fixing the support mandrel on a guiding mandrel by means of an arrangement and an operation different from the latter.

These drawings show interesting but by no means restrictive embodiments, of the intestinal retractor-retainer according to the invention.

This retractor-retainer includes:

a guiding mandrel 1 comprising a proximal portion 2 consisting of a handle shaped to allow it to be fixed, to an operating table, in the desired position, by means of a fastening device 3 which can be of a type known in its own right, and a distal portion 4 of elongated form to be introduced, by means of a trocar T, into abdominal cavity C of a patient during a laparoscopic procedure;

a support mandrel 5 of elongated form; and

a membrane such as a contention net 6.

The device according to the invention still relates to an instrument 7 for the insertion of the support mandrel and the said contention net into the abdominal cavity. This instrument comprises a handle and a rod of which the distal portion at least is hollow to house the said support mandrel and the aforesaid contention net, before introduction into the abdominal cavity.

Guiding mandrel 1 includes, according to a first embodiment shown on FIG. 3, a handle 2 and a tube 8 forming an integral part with handle 2 and arranged coaxially in the extension of the latter.

Terminal part 4a of tube 8 forming distal portion 4 of guiding mandrel 1 is arranged to form the female element to which the distal end of support mandrel 5 is fixed. In tube 8 and handle 2 is fitted, with axial movement capability, a coupling rod 9, the base of which co acts with an operating button 10 accessible through an opening 11 fitted laterally in handle 2. A fixed helicoidal spring 12 positioned, through its opposite ends, on the one hand against the bottom of blind axial passage 13 in handle 2, and on the other, against the upper face of the operating button 10, tends to bring button 10—coupling rod 9 mobile assembly to an active clip-on position. The distal portion 4a of tube 8 is provided with a transverse passage with recess 14 having its axis perpendicular to the axis of coupling rod 9 and across which can move the distal end equipped with a bevel 9a of the latter.

End 4b of distal portion 4 of guiding mandrel 1 presents a rounded form, for example spherical or forming a segment of a sphere, in order to avoid any risk of damaging tissues or organs during handling and installing the aforesaid guiding mandrel.

As an indication, tubular body 8 of the guiding mandrel can be at least 200 mm long and have a maximum external diameter of 5 mm, so as to pass through a trocar of 5. Handle 2 can have a length of more than 150 mm and a diameter of more than 5 mm so that it can be enclosed in a device, known in its own right, fastening it to the operating table and housing button 10, spring 12 and part of rod 9.

Guiding mandrel 1 as described above can be made out of stainless metal, or rigid plastic, or a combination of both.

Support mandrel 5 according to the example of the embodiment illustrated on FIGS. 4 to 6, includes a groove 15 which can be between 80 and 100 mm long and a clip-on head 16 constituting the distal portion of this gutter. The latter has preferably an arc section and thus defines a longitudinal throat 17 for receiving contention net 6 in a folded state.

The rear face of coupling head 16 acts as a stop 16a with circular or other form and diameter or dimensions greater than the bore of the barrel of the insertion instrument in which the support mandrel has to be engaged for insertion into the abdominal cavity, for example 12 mm diameter which is the maximum for such surgical interventions.

The front face of this stop is equipped with a coupling lug 16b, for example of truncated form, equipped with a transverse notch or lug 16c with recessing and constituting the distal end of the support mandrel. In addition, the front face of coupling head 16 has a diametrical cavity 16d of dished form, this cavity being shaped to marry the cylindrical shape of the lateral wall of distal portion 4 of guiding mandrel 1. This arrangement defines one and only one correct position of support mandrel 5 relative to guiding mandrel 1, which largely facilitates handling when coupling the said support mandrel to the said guiding mandrel.

Contention net 6 constituting the contention membrane in the example described here is attached to groove 15 in which it is housed folded ready for introduction into the abdominal cavity.

The connection between support mandrel 5 and contention net 6 depends, amongst other things, on the materials used to manufacture these two elements. Depending on the nature of these materials, the connection may be executed:

by sewing: the mandrel is drilled with holes distributed over the length of the groove; the seam is executed during manufacture using an appropriate thread passing through the holes of the support mandrel and the net meshes;

by bonding;

by heat welding;

or by other methods.

Net 6 is made using an extremely fine and elastic thread, itself executed in a material with the necessary qualities. Other structures, in particular not woven, tight or not, can perform the function of the membrane in a similar way to a net.

It may advantageously have the form shown on FIG. 7 and the dimensions indicated on this figure. This embodiment has a form consisting of: —a large rectangular surface 6A, —a first triangular surface 6B consisting of a right-angled triangle attached on the large side of the right angle to one of small sides of the rectangle, and —a second triangular surface 6C comprising a right-angled triangle attached by the large side of the right angle to a portion of one of the large sides of the aforesaid rectangular surface. The small side of the right angle of first triangle 6B is arranged in the extension of the large sides of rectangle 6A while the small side of the right angle of the second triangle 6C is arranged in the extension on the small side of the rectangle 6A which forms a right angle with the large side of said rectangle extended by the small side of the right angle of the aforesaid first triangle 6B.

For example, main rectangular part 6A can have a 250 mm length and 150 mm width, the sides of the right angle of triangle 6B can have lengths of 150 mm and 90 mm respectively, and the sides of the right angle of the triangle 6C can have lengths of 160 mm and 50 mm, respectively.

Contention net 6 thus formed is fixed to support mandrel 5 via its edge 6D defined by apexes S1 and S2 of triangular surfaces 6B and 6C formed by the angles created by their hypotenuse and the large side of their right angle.

The free edge of contention net 6, in particular when this presents a degree of elasticity, can be reinforced advantageously. In a preferred arrangement, the edge of the net can be reinforced by edge 18 which can be formed by a simple hem sewn using non elastic thread 19 (FIG. 9). In this case, the net is initially cut so as to allow the execution of hem 6a on the edges of the net (FIG. 8).

The dual purpose of this reinforcement is to increase the strength of the contention net:

on the one hand, along its edges which thus have two thicknesses of material, and,

in addition, at the angles or apexes S3, S4 and S5 of the net intended to be attached to the internal abdominal wall using needles and which present, therefore, three thicknesses of material at these places.

It is observed that the material used to constitute the net is extremely thin and elastic. Its thinness is a dominant characteristic because it solves the problem of ultimate withdrawal: at the end of the operation, the net is completely unfolded but is so fine that it can be drawn out through a trocar without any folding whatsoever. On the other hand, its elasticity is a disadvantage because a certain rigidity is required to properly retain the intestines. This is why the thread used to sew the hems overcomes this disadvantage: they are non-elastic and rigidify the net at its edges. To rigidify the central part, threads 19′ following the diagonals of the net (FIG. 9) may be added.

Contention net 6 is obviously folded so that it can be inserted into the barrel of the insertion instrument with support mandrel 5, through a trocar. All folding configurations are within the scope of the invention, such as accordion or roll-up in particular

FIGS. 10A to 10F are diagrammatic views illustrating an advantageous method of folding the net.

FIG. 10A shows the net deployed and fixed by its edge 6D in the groove of the support mandrel.

FIGS. 10B and 10C shown two accordion type folding stages of parts 6A and 6B of the net.

FIG. 10D illustrates that the part 6C is folded back over folded part 6A.

Part 6C is then folded in accordion and this folding is then positioned over the fold of part 6A, as shown in FIG. 10E.

FIG. 10F shows the folding of part 6B which is folded back over part 6A.

This folded form can then be placed in the groove of the support mandrel and inserted, with it, inside the barrel of the insertion instrument for delivery.

As indicated previously, apexes S3, S4, S5 of net 6 are designed to be attached to the internal abdominal wall by means of a tie and needles 20a, 20b, 20c.

According to a characteristic arrangement of the invention, these needles are attached originally to apexes S3, S4, S5 of net 6, which means that when manufacturing the contention net each of its angles or apexes S3, S4, S5 have to be provided with a thread 21 and a needle 20, as shown in the FIG. 7

According to a first embodiment illustrated in FIGS. 11A to 11C, the needles are simply sewn into the thicknesses of the folding; this has the advantage of helping to maintain folded the accordion which tends to unfold.

Needle 20a connected to apex S4 (the latter was used by the surgeon when installing the net) is sewn into the folding of part 6A (FIG. 11B) at the folding stage illustrated in FIG. 10C.

Needle 20b, connected to apex S5, is sewn into the folding of parts 6C and 6A (FIG. 11C) at the process folding stage shown in FIG. 10E, whereas needle 20C connected to apex S3 of the net is sewn into the folding of parts 6B and 6A (FIG. 11C) at the folding stage shown in FIG. 10F.

According to another procedure illustrated on FIG. 12, close to each corner or apex S3, S4, S5, contention net 6 is provided with a small sleeve 22 in which needle 20 can be housed attached by a thread 21 to apexes S3, S4 or respective S5. In this way, there no longer exists any risk of the points of the needles passing through all the folded net and injuring the surrounding tissues. On the other hand, the needles placed in the sleeves cannot be used to hold the fold, since they are not traversing.

Insertion instrument 7 which completes the device according to the invention can be created very simply by a rod 23 of which distal portion 23a is at least is hollow. Preferably, this rod consists of a cylindrical tube, the proximal end of which is attached to a handle 24.

Handle 24—tube 23 assembly creates the approximate shape of a gun; therefore, it is referred to by this word in the description which follows, while the aforementioned tube is called the “barrel”.

Support mandrel 5 and folded contention net 6 attached to it are engaged in distal portion 23a of barrel 23. In this position, stop 16a butts against the distal end of barrel 23, so that coupling head 16 of the support mandrel emerges relative to the said end. This arrangement allows automatic fixing, by clipping on the distal end of the support mandrel on the distal portion of the guiding mandrel, as described in the remainder of this description.

According to the procedure shown, the support mandrel and the thread are held inside the barrel only by the friction caused by their size. The gun can also be equipped with a system dedicated to releasing the support mandrel.

Gun 7 cannot be reused. Barrel 23 can contain a system which prevents its re-use. For example, the barrel can be equipped with a system with double bottom which is released during extraction of the net and which can no longer return to its initial position.

The installation of the net and the support mandrel are the only function of the gun. The surgeon withdraws the latter at the end of the surgical procedure directly through the trocars. After cutting the wire holding it to the wall and releasing the support mandrel from the guiding mandrel, the surgeon catches the net in a zone close to the support mandrel and entirely withdraws it through the trocar using a gripper of a type usually used in vascular surgery.

Below is a description of the procedure using the intestinal retractor-retainer according to the invention.

For instance, in an abdominal aortic restoration procedure using laparoscopic or celioscopic technique, the patient is placed on the operating table in the right lateral decubitus position and Trendelenbourg position at 25°.

In this position, gravity naturally moves the intestines towards the right abdominal wall, which releases the peritoneum and the dissection zone.

Using a first size 5 of trocar T1, guiding mandrel 1 is inserted in abdominal wall P and is fastened outside the said wall, using its handle 2 to the operating table by means of fastener 3 which can be of a type known in its own right. Using another trocar T2 of maximum size 12, support mandrel 5 and net 6 are introduced into abdominal cavity C, by means of gun 7 (FIG. 15). In this insertion phase, the surgeon thus handles this mandrel from outside the abdominal cavity C using gun 7. The surgeon will then bring the distal end of the support mandrel towards the distal portion of the guiding mandrel (FIG. 16A).

During its insertion into transverse passage 14 of the distal portion of guiding mandrel 1, the truncated coupling tenon 16b slides along bevel 9a of blocking rod 9, leading to a displacement towards the top of the aforesaid rod and button 10, against the opposing action of spring 12 (FIG. 16B).

By continuing the introduction of coupling tenon 16b, the mobile blocking rod 9 falls into the transverse notch 6c located to the rear of the said tenon, under the pressure exerted by spring 12 (FIG. 16C). The support mandrel is thus coupled to the distal portion of the guiding mandrel.

It is then possible to withdraw gun 7, while leaving in place support mandrel 5 attached to guiding mandrel 1.

Withdrawal of the gun reveals the net folded on the support mandrel.

The needles and the thread for attaching the net to the abdominal wall are already joined to the net. The surgeon takes the first needle and brings it towards the place on the abdominal wall he wants to pass it through, which deploys the net in part. Similarly for the two other needles.

As indicated previously, needles 20a, 20b, 20c attached to apexes S4, S5, S3, respectively, have to appear in a pre-determined sequence to simplify the installation of the net, after the withdrawal of the gun.

Needle 20c attached to apex S3 should appear first, the surgeon grasps it and takes it through abdominal wall at I, which leads to partial deployment of the net. Then needle 20b connected to apex S5 is brought to the distal point of the root of the mesentery, at II. Needle 20a attached to apex S4 is brought to III.

The net is then completely deployed and secured and the intestinal retractor-retainer according to the invention is installed.

Part 6B of the net retains the transverse colon.

Part 6A of the aforesaid net retains face F.

Part 6C of the latter retains the intestines along the mesentery.

To separate the support mandrel and the guiding mandrel at the end of the surgical operation, pull back button 10 connected to clip-on rod 9 at the level of handle 2 so as to release the aforementioned support mandrel and allow its withdrawal (FIG. 17).

FIGS. 18A to 18D illustrate a second embodiment of the guiding mandrel and method of fixing support mandrel 5 onto it.

On these figures, the component parts of guiding mandrel 1 and support mandrel 5, which are identical or nearly identical to the component parts of the procedure previously described, are shown by the same references.

Axial displacements of coupling rod 9 in tube 8 constituting distal portion 4 of the guiding mandrel are controlled by push-button 25 emerging at the upper end of handle 2 and enabling a mechanism (not shown) housed in the said handle, so that by successively pressing this push-button, either coupling rod 9 end is inserted into transverse passage 14, or the aforesaid end in tube 8 is withdrawn. Such mechanisms, known in their own right, are widely used in the manufacture of certain retractable lead or ball writing tools.

FIG. 18A shows the retracted position of coupling rod 9 in tube 8 of guiding mandrel 1, before engaging coupling lug 16b of support mandrel 5 in the transverse passage 14 placed in distal end 4a of the aforesaid guiding mandrel.

FIG. 18B shows coupling lug 16b of support mandrel 5 engaged in transverse passage 14 of the distal end of guiding mandrel 1, in a position whereby the lug or transverse notch 16c is placed in the alignment of coupling rod 9.

FIG. 18C shows the insertion of the distal end of coupling rod 9 in the notch of coupling head 16 of support mandrel 5, obtained by pressing push-button 25.

FIG. 18D shows the fixing of support mandrel 5 on guiding mandrel 1.

The support mandrel and the guiding mandrel are separated at the end of the surgical operation by pressing on push-button 25. This actuates the mechanism retracting rod coupling 9.

Other means could be used to ensure the rapid or quasi instantaneous joining and separation of the support mandrel and guiding mandrel, for example means using an electromagnetic force.

1. Intestinal retractor-retainer for celioscopic surgery characterized in that it comprises: a guiding mandrel (1) with proximal portion (2) consisting of handle (2) that can be connected to an operating table, a distal portion (4) of elongated form for introduction into the abdominal cavity during laparoscopic interventions; a support mandrel (5) with elongated form on which and along which is fixed a contention membrane, folded according to a folding method allowing its in situ deployment; the distal end of this support mandrel (5) and the distal portion of guiding mandrel (1) being equipped with complementary quick coupling means (14, 9; 16b, 16c); and an instrument (7) for inserting support mandrel (5) and contention membrane into the abdominal cavity and fixing it on the guiding mandrel, this instrument comprising a barrel (23) the distal portion (23c) of which is at least hollow and dimensioned to receive the said support mandrel (5) and contention membrane connected to the latter.

2. Intestinal retractor-retainer, according to claim 1, in which the contention membrane is a contention net (6).

3. Intestinal retractor-retainer according to claim 1, characterized in that the distal portion (4) of guiding mandrel (1) consists of tube (8) in which is placed a coupling rod (9) assembled with an axial movement capability in the said tube (8) whose distal end (4a) is provided with a transverse passage (14), the distal end of support mandrel (5) having a lug (16b) for insertion into the said transverse passage and provided with a notch (16c) in which the distal end of coupling rod (9) can be engaged, so as to fix support mandrel (5) on guiding mandrel (1).

4. Intestinal retractor-retainer according to claim 3 characterized in that coupling rod (9) is subjected to the action of a spring (12) tending to push it back into the coupling position, so that the fixing of support mandrel (5) on guiding mandrel (1) takes place automatically by simply pressing on the coupling lug of the said support mandrel in traverse passage (14) of the guiding mandrel.

5. Intestinal retractor-retainer according to claim 3, characterized in that handle (2) of guiding mandrel (1) contains a mechanism with push-button control (25) so that, by successively pressing on this push-button, the end of coupling rod (9) is either introduced through traverse passage (14), or said end is retracted in tube (8).

6. Intestinal retractor-retainer according to claim 1, characterized in that support mandrel (5) takes the form of a groove (15), for example with a cross section of an arc of circle, in which contention membrane is housed in a folded state, the said membrane being fixed through one of its edges (6D) in the said groove (15).

7. Intestinal retractor-retainer according to claim 1, characterized in that the contention membrane has a form consisting of a rectangular surface (6A), a first triangular surface (6B) consisting of a right angle triangle attached by its large side of the right angle to one of the small sides of the rectangular surface (6A), and a second triangular surface (6C) consisting of a right angle triangle attached by its large side of the right angle to one of the large sides of the rectangular surface (6A), and in that the small side of the right angle of the first triangle (6B) is arranged in the prolongation of one of the large sides of rectangle (6A), whereas the small side of the right angle of the second triangle (6C) is arranged in the prolongation of the small side of rectangle (6A) which forms a right angle with the large side of the said rectangle prolonged by the small side of the right angle of the said first triangle (6B).

8. Intestinal retractor-retainer according to claim 7, characterized in that the contention membrane is fixed to support mandrel (5) through its edge (6D) defined by apexes (S1 and S2) of triangular surfaces (6B, 6C), consisting of the angles formed by their hypotenuse and the large side of the right angle.

9. Intestinal retractor-retainer according to claim 1, characterized in that the free edge of the contention membrane is reinforced.

10. Intestinal retractor-retainer according to claim 9 characterized in that the contention membrane has a degree of elasticity and in which the free edge of the contention membrane is reinforced by an edge (18), preferably consisting of hems sewn using non elastic wires (19).

11. Intestinal retractor-retainer according to claim 1, characterized in that the angles or apexes (S3, S4, S5) of the contention membrane for attaching to the abdominal wall of the patient for execution of the laparoscopic intervention are provided with needles (20a, 20b, 20c) tied by wires (21) to the said angles or apexes.

12. Intestinal retractor-retainer according claim 11, characterized in that the angles or apexes (S3, S4, S5) of the contention membrane are equipped with small sleeves (22) in which are placed needles (20a, 20b, 20c).

13. Intestinal retractor-retainer according to claim 1, characterized in that the contention membrane has been folded in accordion.

14. Intestinal retractor-retainer according to claim 1, characterized in that the contention membrane is rolled up.

15. Intestinal retractor-retainer according to claim 1, characterized in that the distal end of support mandrel (5) consists of a coupling head (16) whose rear face (16a) acts as a stop placed against the distal end of barrel (23) of insertion instrument (7).

16. Intestinal retractor-retainer according to claim 1 characterized in that the front face of coupling head (16) consists of a diametrical cavity (16d) of curved form, this cavity being shaped to marry the cylindrical form of the side wall of the distal portion (4) of guiding mandrel (1).

17. Intestinal retractor-retainer according to claim 4, characterized in that coupling head (16) is provided with a clip-on lug (16b) with truncated form, and that the distal end of mobile coupling rod (9) of guiding mandrel (1) emerges into a transverse passage (14) arranged in the distal portion of guiding mandrel (1) and has a bevel (9a) against which slides the said lug when introduced into the said passage, leading to the upward movement of the said coupling rod.

18. Intestinal retractor-retainer according to claim 1, characterized in that proximal of coupling rod (9) co acts with an operating button (10) which is accessible through an opening (11) arranged laterally in handle (2) of guiding mandrel (1), a spring (12) bearing on the upper face of said button (10) so as to push back coupling rod (9) into coupling position.

19. Intestinal retractor-retainer according to claim 2, characterized in that the distal portion (4) of guiding mandrel (1) consists of tube (8) in which is placed a coupling rod (9) assembled with an axial movement capability in the said tube (8) whose distal end (4a) is provided with a transverse passage (14), the distal end of support mandrel (5) having a lug (16b) for insertion into the said transverse passage and provided with a notch (16c) in which the distal end of coupling rod (9) can be engaged, so as to fix support mandrel (5) on guiding mandrel (1).

20. Intestinal retractor-retainer according to claim 19 characterized in that coupling rod (9) is subjected to the action of a spring (12) tending to push it back into the coupling position, so that the fixing of support mandrel (5) on guiding mandrel (1) takes place automatically by simply pressing on the coupling lug of the said support mandrel in traverse passage (14) of the guiding mandrel.
